(19) United States
(12) Patent Application Publication
(10) Pub. No.: US 2017/0001946 A1
(43) Pub. Date: Jan. 5, 2017

(54) TEREPHTHALATE DERIVATIVES AND COMPOSITIONS THEREOF

(71) Applicant: Sadesh Sookraj, Waltham, MA (US)

(72) Inventor: Sadesh H. Sookraj, Waltham (ZA)

(21) Appl. No.: 15/197,881

(22) Filed: Jun. 30, 2016

Related U.S. Application Data
(60) Provisional application No. 62/188,378, filed on Jul. 2, 2015.

(51) Int. Cl. C07C 69/82; C08G 63/183

(52) U.S. Cl. CPC C07C 69/82; C08G 63/183

(57) ABSTRACT

The present invention provides dimethylterephthalate in which two of the aromatic ring carbons are derived from ethanol. The present invention also provides bis (2-hydroxyethyl) terephthalate, wherein two to six carbon atoms in a molecule can be derived from ethanol.

### CROSS REFERENCE TO RELATED TO APPLICATIONS

The present application claims benefit from U.S. Ser. No. 62/188,378 filed Jul. 2, 2015, which is hereby incorporated by reference in its entirety as if fully restated herein.

### BACKGROUND OF THE INVENTION

Terephthalic acid (TPA) is used in conjunction with isophthalic acid (IPA) to produce polyethylene terephthalate (PET) which is used extensively in consumer goods packaging, most prominently in the now ubiquitous plastic water bottles.

There is strong demand from consumers and consumer goods companies for sustainable alternatives to petroleum-based plastics for packaging applications. Indeed Coca Cola® and others have recently introduced PET based biobased monoethylene glycol (MEG). The resulting bottles are branded as “Plant Bottle™” and have been well received in the marketplace. Unfortunately, since about 70% of the mass in PET derives from terephthalic and isophthalic acids, replacing petroleum-sourced MEG with biobased material yields PET that is only about 30% biobased. There is considerable interest in biobased TPA and IPA, or esters thereof, and PET.

### SUMMARY OF THE INVENTION

The present invention addresses the problem that current biobased routes to terephthalate (such as terephthalic acid, or esters thereof) are carbon inefficient. Ethanol production provides an efficient biobased chemical process, and ethanol can be utilized as a primary feedstock for terephthalate production.

In the ethanol-involved terephthalate production, one of the two ethanol-derived carbon atoms will be the carbon atom in the terephthalate that is bonded to a carboxy group. Alternatively, in the process wherein terephthalate is derived from a reaction of ethylene with dimethylfuran, both of the ethylene-derived carbons are at unsubstituted positions of the aromatic ring.

Accordingly, in one aspect, provided herein is a dimethylterephthalate (DMTA) composition comprising dimethylterephthalate molecules wherein two of the carbon atoms in the aromatic ring of the terephthalate moiety are derived from ethanol.

In another aspect, provided herein is a polymer composition derived from a dimethylterephthalate described herein.

In another aspect, provided herein is a bis (2-hydroxyethyl) terephthalate composition comprising bis (2-hydroxyethyl) terephthalate molecules wherein two of the carbon atoms in the aromatic ring of the terephthalate moiety are derived from ethanol.

Ethanol can be converted to monoethylene glycol. Thus, biobased ethanol can produce biobased monoethylene glycol, wherein both carbons are derived from biobased ethanol. A further reaction between the terephthalic acid and monoethylene glycol can result in a bis (2-hydroxyethyl) terephthalate. Thus, in some embodiments, provided herein is a bis (2-hydroxyethyl) terephthalate composition wherein two of the carbon atoms are derived from biobased ethanol. In some embodiments, provided herein is a bis (2-hydroxyethyl) terephthalate composition wherein four of the carbon atoms are derived from biobased ethanol. In some embodiments, provided herein is a bis (2-hydroxyethyl) terephthalate composition wherein six of the carbon atoms are derived from biobased ethanol.

In another aspect, provided herein is a bis (2-hydroxyethyl) terephthalate composition comprising bis (2-hydroxyethyl) terephthalate molecules characterized in that at least half of the carbon atoms in the molecule are derived from ethanol.

In another aspect, provided herein is a polymer composition derived from a bis (2-hydroxyethyl) terephthalate described herein.

### DEFINITIONS

The term “polymer”, as used herein, refers to a molecule of high relative molecular mass, the structure of which comprises the multiple repetition of units derived, actually or conceptually, from molecules of low relative molecular mass. The term “polymer” further refers to copolymers derived from more than one monomer. Thus, each instance of the term polymer, as used herein, also refers to a copolymer.

Bio-based content: the bio-based content of a material is measured using the ASTM D6866 method, which allows the determination of the bio-based content of materials using radiocarbon analysis by accelerator mass spectrometry, liquid scintillation counting, and isotope mass spectrometry. When nitrogen in the atmosphere is struck by an ultraviolet light produced neutron, it loses a proton and forms carbon that has a molecular weight of 14, which is radioactive. This ¹⁴C is immediately oxidized into carbon dioxide, and represents a small, but measurable fraction of atmospheric carbon. Atmospheric carbon dioxide is cycled by green plants to make organic molecules during photosynthesis. The cycle is completed when the green plants or other forms of life metabolize the organic molecules producing carbon dioxide which is then able to return back to the atmosphere. Virtually all forms of life on Earth depend on this green plant production of organic molecules to produce the chemical energy that facilitates growth and reproduction. Therefore, the ¹⁴C that exists in the atmosphere becomes part of all life forms and their biological products. These renewably based organic molecules that biodegrade to carbon dioxide do not contribute to global warming because no net increase of carbon is emitted to the atmosphere. In contrast, fossil fuel-based carbon does not have the signature radiocarbon ratio of atmospheric carbon dioxide. See WO 2009/155086, incorporated herein by reference.

The application of ASTM D6866 to derive a “bio-based content” is built on the same concepts as radiocarbon dating, but without use of the age equations. The analysis is performed by deriving a ratio of the amount of radiocarbon (¹⁴C) in an unknown sample to that of a modern reference standard. The ratio is reported as a percentage, with the units “pMC” (percent modern carbon). If the material being analyzed is a mixture of present day radiocarbon and fossil carbon (containing no radiocarbon), then the pMC value obtained correlates directly to the amount of bio-based material present in the sample. The modern reference standard used in radiocarbon dating is a NIST (National Institute of Standards and Technology) standard with a known radiocarbon content equivalent approximately to the year AD 1950. The year AD 1950 was chosen because it represented a time prior to thermonuclear weapons testing which introduced large amounts of excess radiocarbon into the atmosphere with each explosion (termed “bomb carbon”). The AD 1950 reference represents 100 pMC. “Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. The distribution of bomb carbon has gradually decreased over time, with today's value being near 107.5 pMC. As a result, a fresh biomass material, such as corn, could result in a radiocarbon signature near 107.5 pMC.

Petroleum-based carbon does not have the signature radiocarbon ratio of atmospheric carbon dioxide. Research has noted that fossil fuels and petrochemicals have less than about 1 pMC, and typically less than about 0.1 pMC, for example, less than about 0.03 pMC. However, compounds derived entirely from renewable resources have at least about 95 percent modern carbon (pMC), they may have at least about 99 pMC, including about 100 pMC.

Combining fossil carbon with present day carbon into a material will result in a dilution of the present day pMC content. By presuming that 107.5 pMC represents present day bio-based materials and 0 pMC represents petroleum derivatives, the measured pMC value for that material will reflect the proportions of the two component types. A material derived 100% from present day biomass would give a radiocarbon signature near 107.5 pMC. If that material were diluted with 50% petroleum derivatives, it would give a radiocarbon signature near 54 pMC.

A bio-based content result is derived by assigning 100% equal to 107.5 pMC and 0% equal to 0 pMC. In this regard, a sample measuring 99 pMC will give an equivalent bio-based content result of 93%.

Assessment of the materials described herein according to the present embodiments is performed in accordance with ASTM D6866 revision 12 (i.e. ASTM D6866-12), the entirety of which is herein incorporated by reference. In some embodiments, the assessments are performed according to the procedures of Method B of ASTM-D6866-12. The mean values encompass an absolute range of 6% (plus and minus 3% on either side of the bio-based content value) to account for variations in end-component radiocarbon signatures. It is presumed that all materials are present day or fossil in origin and that the desired result is the amount of bio-based carbon “present” in the material, not the amount of bio-material “used” in the manufacturing process.

Other techniques for assessing the bio-based content of materials are described in US. Pat. Nos. 3,885,155, 4,427,884, 4,973,841, 5,438,194, and 5,661,299, and WO 2009/155086, each of which is incorporated herein by reference.

### DETAILED DESCRIPTION OF THE INVENTION

### Conversion Schemes

Schemes 1-3 below depict exemplary conversion schemes for preparing composition described herein.

Scheme 1 depicts conversions including that of ethanol to ethylene oxide, beta propiolactone, acrylic acid and/or maleic anhydride, and terephthalic acid (i.e., bio TPA) via, for example, the known Henkel process.

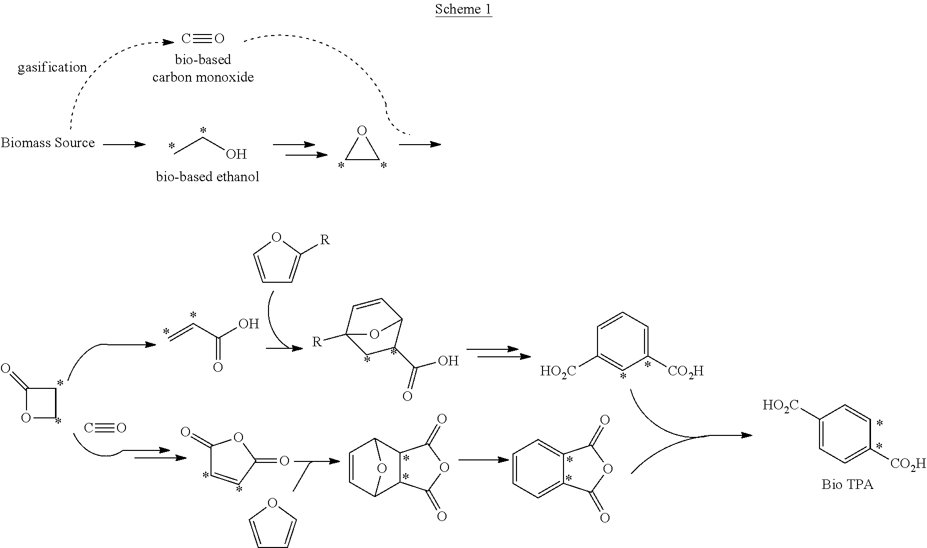

Scheme 2 depicts the conversion of bio TPA to DMTA and/or bis(2-hydroxyethyl)terephthalate.

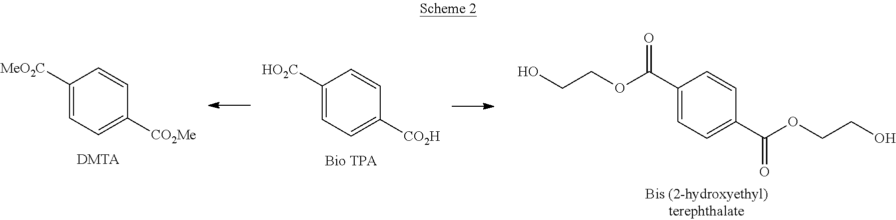

Scheme 3 depicts the conversion of ethanol to ethylene oxide and monoethylene glycol (MEG), which is combined with bio-TPA to make bio-PET.

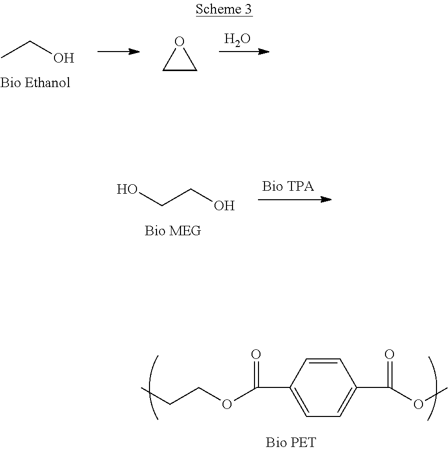

Methods of making beta propiolactone from the carbonylation of ethylene oxide are known in the art and include those described in WO 2013/063191 and WO 2014/004858.

Methods of making succinic anhydride from the carbonylation of ethylene oxide are known in the art and include those described in WO 2012/030619 and WO 2013/122905. Succinic anhydride is oxidized to maleic anhydride by known methods.

Methods of making acrylic acid from beta propiolactone are known in the art and include those described in WO 2013/126375, WO 2010/118128 and WO 2013/063191. The entire contents of each of the above publications is hereby incorporated by reference.

### DMTA and Polymer Compositions Thereof

In one aspect, the present invention provides a dimethylterephthalate composition comprising dimethylterephthalate molecules wherein two of the carbon atoms in the aromatic ring of the terephthalate moiety are derived from ethanol.

In some embodiments, one of the ethanol-derived carbon atoms in the aromatic ring is directly bonded to a carboxymethyl group.

In some embodiments, one of the ethanol-derived carbon atoms in the aromatic ring is directly bonded to a hydrogen atom.

In some embodiments, the two ethanol-derived carbon atoms in the aromatic ring are adjacent to each other in the ring. In some embodiments, one of the two ethanol-derived carbon atoms in the aromatic ring is directly bonded to a carboxymethyl group. In some embodiments, the ethanol-derived carbon atoms in the aromatic ring are not both directly bonded to hydrogen atoms.

In some embodiments, the composition contains a mixture of dimethylterephthalate molecules that differ with respect to the position in the aromatic rings of the ethanol-derived carbon atoms.

In some embodiments, the dimethylterephthalate molecules having two ethanol-derived carbon atoms in the aromatic ring comprise at least 10% of all dimethylterephthalate molecules in the composition.

In some embodiments, the dimethylterephthalate molecules having two ethanol-derived carbon atoms in the aromatic ring comprise at least 20%, at least 30%, at least 50%, at least 75%, or at least 90% of all dimethylterephthalate molecules in the composition.

In some embodiments, the ethanol is derived from a biological source (i.e., a biobased ethanol). In some embodiments, the biobased ethanol has a biobased content of 100%. In some embodiments, the biobased ethanol has a pMC of 107.5.

In some embodiments, provided herein is a dimethylterephthalate composition comprising dimethylterephthalate molecules wherein two of the carbon atoms in the aromatic ring of the terephthalate moiety are derived from ethanol, and wherein at least one dimethylterephthalate molecule has a pMC of greater than zero. In some embodiments, the at least one dimethylterephthalate molecule has a pMC of between zero and about 21.5. In some embodiments, the at least one dimethylterephthalate molecule has a pMC of at least about 21.5.

In some embodiments, provided herein is a dimethylterephthalate composition comprising dimethylterephthalate molecules wherein two of the carbon atoms in the aromatic ring of the terephthalate moiety are derived from ethanol, and wherein at least one dimethylterephthalate molecule has a biobased content of greater than zero. In some embodiments, the at least one dimethylterephthalate molecule has a biobased content of between zero and about 20%. In some embodiments, the at least one dimethylterephthalate molecule has a biobased content of at least about 20%.

In some embodiments, the carboxy carbon atoms of a dimethylterephthalate molecule are derived from carbon monoxide that is present in the terephthalate production.

In another aspect, provided herein is a polymer composition derived from a dimethylterephthalate composition described herein.

Bis (2-hydroxyethyl) Terephthalate and Polymer Compositions Thereof

In one aspect, provided herein is a bis (2-hydroxyethyl) terephthalate composition comprising bis (2-hydroxyethyl) terephthalate molecules wherein two of the carbon atoms in the aromatic ring of the terephthalate moiety are derived from ethanol.

In some embodiments, one of the ethanol-derived carbon atoms in the aromatic ring of a bis (2-hydroxyethyl) terephthalate molecule is directly bonded to a carboxy(2-hydroxyethyl) group.

In some embodiments, one of the ethanol-derived carbon atoms in the aromatic ring is directly bonded to a hydrogen atom.

In some embodiments, the two ethanol-derived carbon atoms in the aromatic ring are adjacent to each other in the ring. In some embodiments, one of the two ethanol-derived carbon atoms in the aromatic ring is directly bonded to a carboxy(2-hydroxyethyl) group. In some embodiments, the ethanol-derived carbon atoms in the aromatic ring are not both directly bonded to hydrogen atoms.

In some embodiments, the composition contains a mixture of bis (2-hydroxyethyl) terephthalate molecules that differ with respect to the position of the ethanol-derived carbon atoms in the aromatic rings.

In some embodiments, the bis (2-hydroxyethyl) terephthalate molecules having two ethanol-derived carbon atoms in the aromatic ring comprise at least 10% of all bis (2-hydroxyethyl) terephthalate molecules in the composition.

In some embodiments, the bis (2-hydroxyethyl) terephthalate molecules having two ethanol-derived carbon atoms in the aromatic ring comprise at least 20%, at least 30%, at least 50%, at least 75%, or at least 90% of all bis (2-hydroxyethyl) terephthalate molecules in the composition.

In some embodiments, the bis (2-hydroxyethyl) terephthalate composition comprises hydroxyethyl moieties derived from ethanol.

In some embodiments, the ethanol is derived from a biological source (i.e., a biobased ethanol). In some embodiments, the biobased ethanol has a biobased content of 100%. In some embodiments, the biobased ethanol has a pMC of 107.5.

In another aspect, provided herein is a bis (2-hydroxyethyl) terephthalate composition comprising bis (2-hydroxyethyl) terephthalate molecules characterized in that at least half of the carbon atoms in the molecule are derived from ethanol.

In some embodiments, the bis (2-hydroxyethyl) terephthalate composition comprises bis (2-hydroxyethyl) terephthalate molecules wherein two of the carbon atoms in the aromatic ring of the terephthalate moiety are derived from ethanol.

In some embodiments, one of the ethanol-derived carbon atoms in the aromatic ring of a bis (2-hydroxyethyl) terephthalate molecule is directly bonded to a carboxy(2-hydroxyethyl) group.

In some embodiments, one of the ethanol-derived carbon atoms in the aromatic ring of a bis (2-hydroxyethyl) terephthalate molecule is directly bonded to a hydrogen atom.

In some embodiments, the two ethanol-derived carbon atoms in the aromatic ring of a bis (2-hydroxyethyl) terephthalate molecule are adjacent to each other in the ring. In some embodiments, one of the two ethanol-derived carbon atoms in the aromatic ring of a bis (2-hydroxyethyl) terephthalate molecule is directly bonded to a carboxy(2-hydroxyethyl) group. In some embodiments, the ethanol-derived carbon atoms in the aromatic ring of a bis (2-hydroxyethyl) terephthalate molecule are not both directly bonded to hydrogen atoms.

In some embodiments, a bis (2-hydroxyethyl) terephthalate composition contains a mixture of bis (2-hydroxyethyl) terephthalate molecules that differ with respect to the position of the ethanol-derived carbon atoms in the aromatic rings.

In some embodiments, the bis (2-hydroxyethyl) terephthalate molecules having at least one half ethanol-derived carbon atoms comprise at least 10% of all bis (2-hydroxyethyl) terephthalate molecules in the composition.

In some embodiments, the bis (2-hydroxyethyl) terephthalate molecules having at least one half ethanol-derived carbon atoms comprise at least 20%, at least 30%, at least 50%, at least 75%, or at least 90% of all bis (2-hydroxyethyl) terephthalate molecules in the composition.

In some embodiments, the ethanol is derived from a biological source (i.e., a biobased ethanol). In some embodiments, the biobased ethanol has a biobased content of 100%. In some embodiments, the biobased ethanol has a pMC of 107.5.

In some embodiments, provided herein is a bis (2-hydroxyethyl) terephthalate composition comprising bis (2-hydroxyethyl) terephthalate molecules wherein two of the carbon atoms in the molecules (e.g., two carbon atoms of the aromatic ring of the terephthalate moiety) are derived from ethanol, and wherein at least one bis (2-hydroxyethyl) terephthalate molecule has a pMC of greater than zero. In some embodiments, the at least one bis (2-hydroxyethyl) terephthalate molecule has a pMC of between zero and about 17.9. In some embodiments, the at least one bis (2-hydroxyethyl) terephthalate molecule has a pMC of at least about 17.9. In some embodiments, the at least one bis (2-hydroxyethyl) terephthalate molecule has a pMC of between about 17.9 and about 35.8. In some embodiments, the at least one bis (2-hydroxyethyl) terephthalate molecule has a pMC of at least about 35.8. In some embodiments, the at least one bis (2-hydroxyethyl) terephthalate molecule has a pMC of between about 35.8 and about 53.7. In some embodiments, the at least one bis (2-hydroxyethyl) terephthalate molecule has a pMC of at least about 53.7.

In some embodiments, provided herein is a bis (2-hydroxyethyl) terephthalate composition comprising bis (2-hydroxyethyl) terephthalate molecules wherein two of the carbon atoms in the molecules (e.g., two carbon atoms of the aromatic ring of the terephthalate moiety) are derived from ethanol, and wherein at least one bis (2-hydroxyethyl) terephthalate molecule has a biobased content of greater than zero. In some embodiments, the at least one bis (2-hydroxyethyl) terephthalate molecule has a biobased content of between zero and about 16.7%. In some embodiments, the at least one bis (2-hydroxyethyl) terephthalate molecule has a biobased content of at least about 16.7%. In some embodiments, the at least one bis (2-hydroxyethyl) terephthalate molecule has a biobased content of between 16.7% and about 33.4%. In some embodiments, the at least one bis (2-hydroxyethyl) terephthalate molecule has a biobased content of at least about 33.4%. In some embodiments, the at least one bis (2-hydroxyethyl) terephthalate molecule has a biobased content of between 33.4% and about 50%. In some embodiments, the at least one bis (2-hydroxyethyl) terephthalate molecule has a biobased content of at least about 50%.

In some embodiments, the carboxy carbon atoms of a dimethylterephthalate molecule are derived from carbon monoxide that is present in the terephthalate production.

In another aspect, provided herein is a polymer composition derived from a bis (2-hydroxyethyl) terephthalate composition described herein.

What is claimed is:

1. A dimethylterephthalate composition comprising dimethylterephthalate molecules wherein two of the carbon atoms in the aromatic ring of the terephthalate moiety are derived from ethanol.

2. The dimethylterephthalate composition of claim 1, wherein one of the ethanol-derived carbon atoms in the aromatic ring is directly bonded to a carboxymethyl group, or a hydrogen atom.

3. The dimethylterephthalate composition of claim 1, wherein the two ethanol-derived carbon atoms in the aromatic ring are adjacent to each other in the ring.

4. The dimethylterephthalate composition of claim 3, wherein one of the two ethanol-derived carbon atoms in the aromatic ring is directly bonded to a carboxymethyl group.

5. The dimethylterephthalate composition of claim 1, wherein the composition contains a mixture of dimethylterephthalate molecules that differ with respect to the position in the aromatic rings of the ethanol-derived carbon atoms.

6. The dimethylterephthalate composition of claim 1, wherein the dimethylterephthalate molecules having two ethanol-derived carbon atoms in the aromatic ring comprise at least 10, at least 20%, at least 30%, at least 50%, at least 75%, or at least 90% of all dimethylterephthalate molecules in the composition.

7. The dimethylterephthalate composition of claim 1, wherein the ethanol is derived from a biological source.

8. A bis (2-hydroxyethyl) terephthalate composition comprising bis (2-hydroxyethyl) terephthalate molecules wherein two of the carbon atoms in the aromatic ring of the terephthalate moiety are derived from ethanol.

9. The bis (2-hydroxyethyl) terephthalate composition of claim 8, wherein one of the ethanol-derived carbon atoms in the aromatic ring is directly bonded to a carboxy(2-hydroxyethyl) group or to a hydrogen atom.

10. The bis (2-hydroxyethyl) terephthalate composition of claim 8, wherein the two ethanol-derived carbon atoms in the aromatic ring are adjacent to each other in the ring.

11. The bis (2-hydroxyethyl) terephthalate composition of claim 8, wherein the composition contains a mixture of bis (2-hydroxyethyl) terephthalate molecules that differ with respect to the position of the ethanol-derived carbon atoms in the aromatic rings.

12. The bis (2-hydroxyethyl) terephthalate composition of claim 8, wherein the bis (2-hydroxyethyl) terephthalate molecules having two ethanol-derived carbon atoms in the aromatic ring comprise at least 10%, at least 20%, at least 30%, at least 50%, at least 75%, or at least 90% of all bis (2-hydroxyethyl) terephthalate molecules in the composition.

13. The bis (2-hydroxyethyl) terephthalate composition of claim 8, comprising hydroxyethyl moieties derived from ethanol.

14. The bis (2-hydroxyethyl) terephthalate composition of claim 1 wherein at least half of the carbon atoms in the molecule are derived from ethanol.

15. The bis (2-hydroxyethyl) terephthalate composition of claim 14, wherein one of the ethanol-derived carbon atoms in the aromatic ring is directly bonded to a carboxy(2-hydroxyethyl) group or to a hydrogen atom.

16. The bis (2-hydroxyethyl) terephthalate composition of claim 14, wherein the two ethanol-derived carbon atoms in the aromatic ring are adjacent to each other in the ring.

17. The bis (2-hydroxyethyl) terephthalate composition of claim 16, wherein one of the two ethanol-derived carbon atoms in the aromatic ring is directly bonded to a carboxy(2-hydroxyethyl) group.

18. The bis (2-hydroxyethyl) terephthalate composition of claim 16, wherein the ethanol-derived carbon atoms in the aromatic ring are not both directly bonded to hydrogen atoms.

19. The bis (2-hydroxyethyl) terephthalate composition of claim 14, wherein the composition contains a mixture of bis (2-hydroxyethyl) terephthalate molecules that differ with respect to the position of the ethanol-derived carbon atoms in the aromatic rings.

20. The bis (2-hydroxyethyl) terephthalate composition of claim 14, wherein the bis (2-hydroxyethyl) terephthalate molecules having at least one half ethanol-derived carbon atoms comprise at least 10%, at least 20%, at least 30%, at least 50%, at least 75%, or at least 90% of all bis (2-hydroxyethyl) terephthalate molecules in the composition.

21. A polymer composition derived from a dimethylterephthalate molecules wherein two of the carbon atoms in the aromatic ring of the terephthalate moiety are derived from ethanol.

22. The polymer composition of claim 21, wherein one of the ethanol-derived carbon atoms in the aromatic ring is directly bonded to a carboxymethyl group, or a hydrogen atom.

23. The polymer composition of claim 21, wherein the two ethanol-derived carbon atoms in the aromatic ring are adjacent to each other in the ring.

24. The polymer composition of claim 23, wherein one of the two ethanol-derived carbon atoms in the aromatic ring is directly bonded to a carboxymethyl group.

25. The dimethylterephthalate composition of claim 21, wherein the composition contains a mixture of dimethylterephthalate molecules that differ with respect to the position in the aromatic rings of the ethanol-derived carbon atoms.
